(19) United States
(12) Patent Application Publication
(10) Pub. No.: US 2021/0012426 A1
(43) Pub. Date: Jan. 14, 2021

(54) METHODS AND SYSTEMS FOR ANAMOLY DETECTION IN DENTAL INSURANCE CLAIM SUBMISSIONS

(71) Applicant: NovoDynamics, Inc., Ann Arbor, MI (US)

(72) Inventors: Mark Brooks, Gregory, MI (US); Sean McMillan, Ann Arbor, MI (US); David Rock, Saline, MI (US); Steven Schlosser, Gregory, MI (US)

(73) Assignee: NovoDynamics, Inc., Ann Arbor, MI (US)

(21) Appl. No.: 16/905,739

(22) Filed: Jun. 18, 2020

Related U.S. Application Data
(60) Provisional application No. 62/871,584, filed on Jul. 8, 2019.

(51) Int. Cl. G06Q 40/08; G06F 16/215; G06Q 10/10; G16H 30/20; G16H 30/40; G16H 50/70; G16H 70/20; G06Q 30/00; A61B 6/14

(52) U.S. Cl. CPC G06Q 40/08; G06F 16/215; G06Q 10/10; G16H 30/20; A61B 6/14; G16H 50/70; G16H 70/20; G06Q 30/0185; G16H 30/40

(57) ABSTRACT

A method is performed on a processor for detecting duplication anomalies in a set of patient dental insurance records submitted as part of a dental insurance claim. At least one hash code is generated for at least some of the patient dental insurance record documents. A Hamming Distance is calculated by comparing the hash code(s) of recently submitted patient dental insurance record documents against a database which includes hash codes generated from previous dental insurance claims. Those dental insurance forms are flagged for further human review if the calculated Hamming Distance between the compared hash codes is less than a threshold amount.

### CROSS-REFERENCE TO RELATED APPLICATIONS

This application claims the benefit of U.S. Provisional No. 62/871,584 (Attorney Docket No. 45154-704.101), filed Jul. 8, 2019, the entire content of which is incorporated herein by reference.

### BACKGROUND OF THE INVENTION

### 1. Field of the Invention

The present invention relates generally to methods and systems for screening and managing dental insurance claim forms. More particularly, the present invention relates to methods and systems for detecting duplications and other anomalies among large numbers of dental insurance claim forms.

Healthcare expenditures in the United States are currently very large and expected to rise as the population ages and treatment options increase. Recent CMS reports set 2017 healthcare spending at $3.5 trillion and 2026 spending estimates at $5.7 trillion, which represents an annual growth rate of 5% over the next decade.

With spending at these levels, it is not surprising that healthcare insurance fraud occurs with significant frequency. In 2012, a RAND Corporation study estimated 10% (or $98 billion) of Medicare and Medicaid expenditures were paid to fraudulent claimants. Applying this rate to the entire health system, shows this level of fraud would amount to $350 billion or more in 2018 despite ongoing efforts by the Federal Government to reduce or eliminate insurance fraud.

National dental expenditures are an important component of overall healthcare spending in the United States. The American Dental Association (ADA) reported in 2017 that dental spending was $124 billion or about 3.7% of all healthcare spending. As with overall healthcare spending, dental insurance fraud is a continuing problem, one estimated to occur at a rate near that of overall healthcare insurance fraud. Assuming a dental insurance fraud rate of 8%, fraudulent claims would amount to about $10 billion in 2018. With loss amounts this large, additional efforts to combat insurance fraud are warranted.

Dental insurance fraud can take a variety of forms, including billing for services not performed; billing for services that are not necessary; up-coding procedures to receive overpayment; altering dates of service to obtain coverage; unbundling or improper use of codes; and, of most interest to the present invention, falsifying patient identities and records to obtain payments for patients who have not received services.

While dental insurers have developed automated assessment procedures to reduce losses due to fraud, these procedures must still rely on expert clinicians to assess image information (e.g., radiographs and photographs.

It is therefore an object of the present invention to provide computer-implemented and other automated approaches to extracting information from dental images that can be used improve current insurer claim assessment procedures. In particular, it would be useful to provide methods and tools for the automated analysis of dental images to assist in detecting fraudulent dental insurance claims.

### 2. Description of the Background Art

U.S. Pat. No. 9,940,677 describes a computer-implemented system and method for detecting property insurance fraud. U.S. Pat. No. 9,710,599 describes a computer-implemented system and method for managing radiographic images being assessed as part of insurance claims. See also, US20180039733 and US20100145734.

### SUMMARY OF THE INVENTION

The present invention provides computer-assisted methods and systems for helping to identify fraudulent dental insurance claims. In particular, the methods and systems of the present invention can lessen the need to rely on expert clinicians to evaluate dental insurance claims 1) by using computer-implemented tools to assess image-based dental insurance claim information; (2) by increasing the number of dental insurance claims that can have their image-based information assessed in a cost-effective manner; (3) by increasing the fraud detection rate; and, (4) by increasing the efficiency of expert clinician review by prioritizing claims for review.

In a first aspect, the present invention provides a method for detecting duplication anomalies in a set of patient dental insurance records submitted as part of a dental insurance claim. The method will be performed on a processor where the processor will be associated with a reference database which contains information derived from dental insurance records previously submitted with prior dental insurance claims from a population of patients. The processor and the database may be co-located in a common facility or installation in order to maintain patient privacy, but in other instances may be separated or distributed among two, three, four, five or more locations and/or have at least portions of the processor capability and/or the reference database storage located in the cloud.

Patient dental insurance records typically contain numerous documents and may include, but are not limited to, images of a patient's teeth, patient probe depth charts, patient correspondence, and the like. Patient dental insurance records may be submitted by a dentist, patient, or other submitting party in a digital or non-digital form. Non-digital records are typically scanned or otherwise digitized for evaluation and processing by the methods described herein.

Once the patient records have been received in or converted to a digitized form, a hash code will be generated from the digitized image. If a digitized image contains multiple views of a patient's teeth, in addition to the original image, the individual views are extracted from the image as separate images, and a hash code is generated for each image.

The hash codes are then compared against a database which includes hash codes generated from previous dental insurance claims. If two hash codes are identical or determined to be sufficiently similar by calculating a Hamming distance score, the dental records are flagged as anomalies and additional screening of the full records is performed. To be flagged as needing further screening in accordance with the present invention, the calculated Hamming distance score between two hash codes must reach a minimum threshold value and the minimum threshold value may be adjusted based on anomaly selection criteria, such as the success rate at which the automated screening is able to identify fraudulent records and/or the failure rate at which the automated screening flags legitimate patient claims for further screening. Flagging may comprise identifying only the anomalous patient insurance claim or identifying both the anomalous patient insurance claim and the records represented by the hash code in the reference data base with the Hamming distance below the defined threshold value.

It will be appreciated that once the hash codes have been generated, those scores are then incorporated into the reference database so that future patient record submissions can be compared against a continuously updated reference database.

To reduce the time required to compare two hash codes, some patient record images or extracted teeth views may be classified, and the classification information may be stored in a reference database and associated with its corresponding hash code. For example, a patient record image may be classified as a patient depth chart, a radiograph, correspondence or the like. Extracted teeth views may be classified as a bitewing image, a periapical image, a panoramic image or the like. Hash code comparison time between recently submitted patient dental insurance records and the reference database is reduced if only hash codes from similarly classified images are compared.

In a second aspect, the present invention provides methods for establishing and maintaining a reference database including hash codes representing sets of historic patient dental insurance records submitted in support of previous dental insurance claims. The hash codes may be used for comparison against future patient dental records which have been processed similarly to generate hash codes.

In a third aspect of the present invention, reference databases are provided which comprise a plurality of hash codes representing a plurality of sets of patient dental insurance records submitted as part of a plurality of dental insurance claims. The patient database may be maintained on a server, in the cloud, or in any other hardware system that allows maintenance and periodic updating of the data in the database. The hash codes may be generated by any of the methods and processes described elsewhere herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flow chart setting forth the steps of the present invention for constructing a reference database.

FIG. 2 represents a teeth view extraction step of the methods of the present invention.

FIG. 3 illustrates a, exemplary Hamming Scorecard generated as part of the methods of the present invention.

FIG. 4 is a flow chart setting forth the steps of the present invention for computing and comparing hash codes from the image data.

### DETAILED DESCRIPTION OF THE INVENTION

The present invention provides a system or “suite” of computer-implemented tools which detect and identify dental insurance claims that are anomalous in one or more ways which indicate that they may be fraudulent. The anomaly detection software system of the present invention will be referred to herein as AD. The following sections describe a single anomaly detection tool that is designed to identify image data that is submitted in conjunction with more than one insurance claim. While there are situations in which multiple submissions of image data may be appropriate, in many or most such situations such multiple submissions can be an indication that a claim is fraudulent. For example, when fraudulently billing for services not performed, a dental provider may submit supporting radiographs taken from the record a different patient for whom the radiographs have been previously submitted. Therefore, it is important to be able to automatically identify such duplicate submissions and flag them for further consideration. This anomaly detection tool is referred to as ND-AD #1, or simply, AD #1 in the discussion below.

### Anomaly Detection #1 (AD #1): Duplicate Documents

A dental insurance claim submission typically includes ADA-approved form data that identifies the patient, the provider, the procedure(s) claimed and the reimbursement requested as well as discretionary supporting documentation such as radiographs, photographs, probe charts and letter correspondence. Typically, these claim elements are unique although they need not be. As noted earlier, the submission of duplicate documents in conjunction with multiple insurance claims can signify fraud and are, therefore, regarded as an anomaly requiring detection and further evaluation.

As a computer processing task, the detection of duplicate documents is straight-forward. It calls for the use of a reference database comprising all documents seen previously with which incoming documents can be compared. When a document is an exact replica of a previously submitted document, a bit-for-bit comparison of the digital files resolves the question of uniqueness. In practical implementations of the comparison process, in which the reference document database may contain millions of document representations, efficient storage and comparison operations are essential. To achieve the required efficiencies, the AD #1 algorithm represents documents as hash codes. A hash code representation of a document maps a document's bit-level description of arbitrary size to a (specified) fixed-length bit string (i.e., hash code). By design, the method is deterministic so the same document always maps to the same string and small changes in the document result in large changes in the hash code. Hash codes can be compared to determine if they came from the same source document or to estimate the degree of similarity of the source documents.

The need for similarity comparisons between insurance claim documents (rather than exact comparisons) arises in a variety of natural ways since different submissions of the same documents may be visually distinct for many reasons: visual changes due to photocopying (with resulting scale and contrast changes), handwritten annotation, highlighter markup, and physical modification (key portions cut out with scissors). Hash code algorithms provide degree-of-similarity comparisons between documents that “ignore” many typical document distortions, thereby making it possible to automate the search for duplicate documents over a range of real-world document variations.

The next two sections summarize how an appropriate reference database can be created and how it is used to determine whether newly submitted claim documents are either exact duplicates of a prior submission or are sufficiently similar that further consideration by a claim reviewer is called for. The AD #1 algorithm contains parameters that can be adjusted to precisely define the meaning of “sufficiently similar” in the context of an insurers claim review process. In other words, the AD #1 algorithm can be adjusted to support the business logic required by a particular review process.

### AD #1 Reference Database Construction

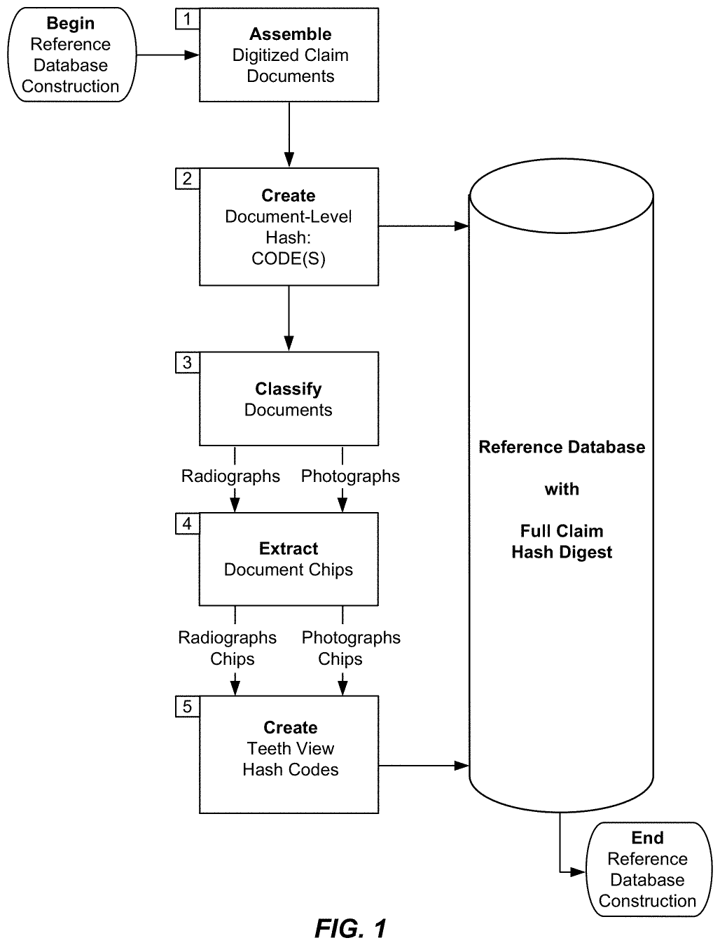

The diagram in FIG. 1 outlines the major steps in the construction of the AD #1 reference database. Each numbered step is individually described to further clarify the overall process. The section concludes with a comment on the need for efficient operation of the database storage and image matching mechanisms

Step C1: Assemble Digitized Claim Documents. To simplify the description of the construction process, it is assumed here that a complete insurance claim includes the following: (1) an ADA-approved claim form, (2) one or more radiographs, (3) one or more photographs, (4) one or more letters of correspondence, and (5) one or more probe charts. Each of these document types enters the construction process in digitized form, having been created by the provider, scanned by the insurance company or, more often, a dental claim clearinghouse (e.g., Apex).

The reference database is based on a schema that includes, at a minimum, the following information: (1) dental provider ID, (2) patient ID, (3) claim ID, and (4) hash keys derived from the submitted claim documents.

Step C2: Create Document-Level Hash. Each page of each document type is encoded as a bit-string (hash code). The role of hash codes is summarized in the following section (Section D).

Step C3: Classify Documents. Determining that an exact duplicate document page has been submitted as part of a claim, given a reference database, is straight-forward. Much more challenging is determining that a duplicate page has been submitted when the original has been modified in ways that alter the page's visual appearance but leave its insurance-related information content unchanged. This may happen, as previously noted, through photocopying or handwritten annotations such as side notes, underlining or strikeouts.

To address this more challenging aspect of duplicate detection, it is necessary to categorize the incoming document stream so claim elements are known as radiographs, probe charts, photographs or other (e.g., letters of correspondence or other miscellaneous material). This classification of the document stream is carried out on a page-by-page basis using proprietary neural network classifiers.

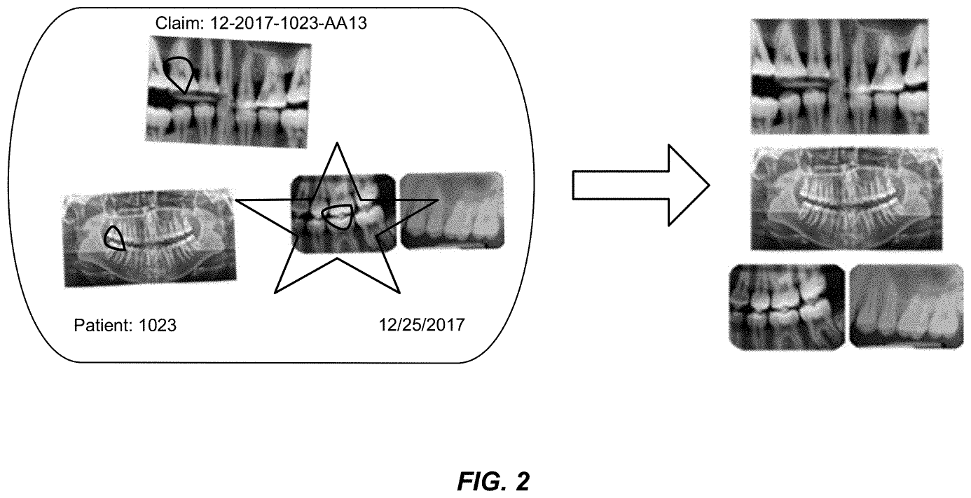

Step C4: Extract Individual Teeth Views. The teeth view extraction process is organized to separate and normalize each view from any extraneous data or stray markings so that only its information content is encoded in the hash code process of Step 5. A common case where this type of processing is needed can be seen in FIG. 2. Here a provider has assembled three radiographs in different orientations and then photocopied the set after adding some annotation. In this case, teeth view extraction requires that the page be identified as one containing radiographs (done in Step 3), that the regions corresponding to only the individual radiographs be identified, separated/segmented and normalized (i.e., rotated into standard orientation for viewing, with brightness and contrast adjustments made when appropriate).

Teeth view extraction for photographs proceeds similarly. The teeth view extraction processes are based on proprietary image analysis algorithms developed by NovoDynamics.

Step C5: Create Teeth View-Level Hash. Each radiographic and photographic teeth view is converted into a hash code and entered into the database as part of the claim record. Efficient database storage and image matching operations are required for practical implementations of this anomaly detection method. It's clear from the above description that the AD #1 Reference Database contains far more items than the quantity of individual claims submitted for review and reimbursement. For example, a 2-page claim form accompanied by 4 bitewing radiographs, that each result in 2 radiograph teeth views, produces 14 database items. Consequently, an insurer that processes, say, 50 million claims annually, could expect its Reference Database to grow to several hundred million items in one year, and perhaps a billion items over a five-year period. Fortunately, efficient methods of searching hash tables of the size required by the Reference Database exist. The AD #1 algorithm incorporates a highly efficient search function based on the concept of multi-index hashing. A representative description of this approach can be found in Punjani (2012) (A. Punjani. Fast Search in Hamming Space with Multi-index Hashing. Proceedings of the 2012 IEEE Conference on Computer Vision and Pattern Recognition (CVPR).

### Operation of the AD #1 Algorithm

Duplicate document searches match the hash codes of incoming documents with those of prior documents both at the level of full documents and at the level of individual teeth views. That is, given a new claim, each hash code is computed for each page of each new document and all accompanying teeth views. Then a Hamming distance is computed between the hash codes of the new pages and teeth views and the hash codes of all pages and teeth views in the database. The hash functions employed in this application map each document into a bit-string of a single length. In that case, the Hamming distance between two documents is equal to the number of positions at which the bit-strings differ. Exact matches have a Hamming distance of zero while inexact matches are greater than zero. Depending on the degree of sensitivity desired in the (inexact) matching process, different Hamming distance thresholds can be set. The business logic employed by an insurer can be used to establish appropriate thresholds for their anomaly detection process.

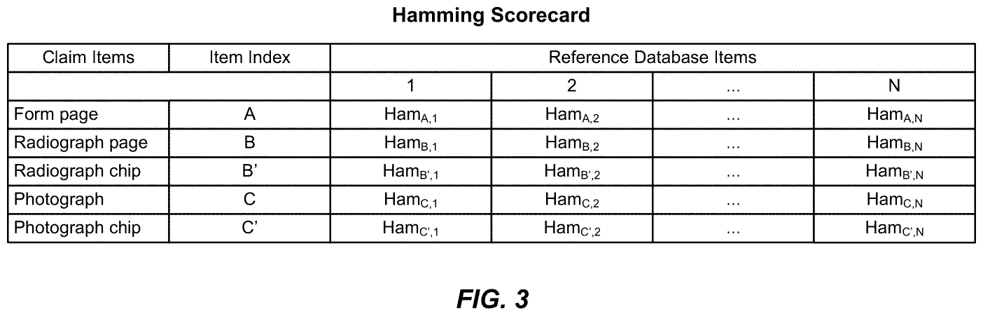

To exemplify the Hamming computation described above, consider the case of a claim that consists of a one-page dental claim form (line item A in the Scorecard table), one radiograph (item B) that produces a single radiograph teeth view (item B′) and one photograph that produces a single photograph teeth view (item C′). See FIG. 3. Then all possible Hamming distances between each new claim element and all Reference Database items from 1 to N, where N denotes the number of items in the database. Table entries HamA,1, . . . , HamC′,N represent the Hamming distances determined. This array of values constitutes what we call the Hamming Scoreboard (for an incoming claim and a given database state). Formation of the Hamming scoreboard is an essential step in the operation of the AD#1 algorithm.

This example Scoreboard calculation extends naturally to the most general case where the claim elements (form, radiograph, photograph) consist of one or more pages. In that situation, the table is expanded by adding a new row for each additional page and calculating the added Hamming distances.

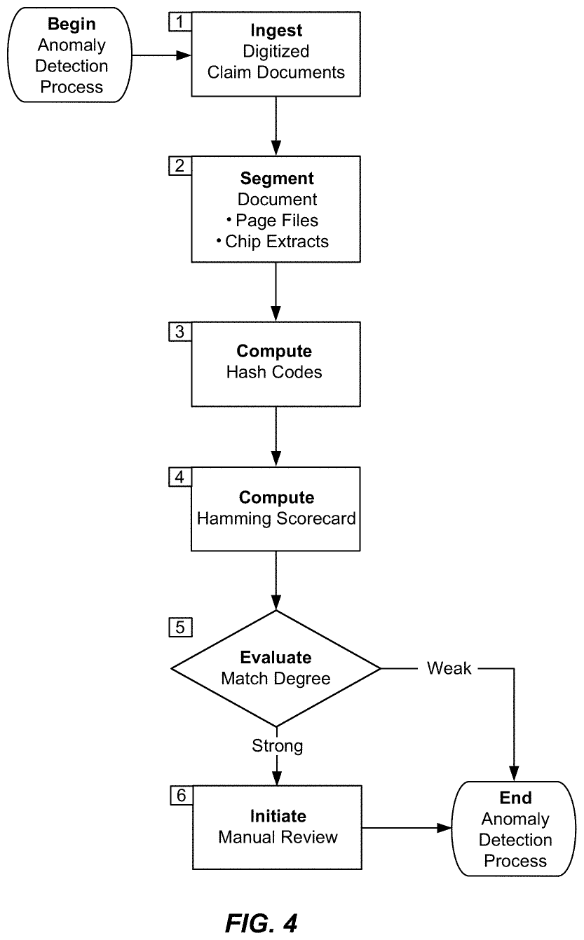

With the concept of the Hamming scoreboard in mind, it is straightforward to outline the operation of the AD #1 anomaly detection algorithm (FIG. 4). Each numbered step is briefly described to further clarify the overall process.

Step D1: Ingest Claim Documents. New claims, in general, may consist of digitized dental claim forms, radiographs, and photographs as well as other supporting material such as probe charts and letters of correspondence. The digitized items are in one or more standard image formats such as PDF, JPEG, PNG or TIFF. PDFs may contain embedded image files. Here we assume the document stream consists of only claim forms, radiographs and photographs, perhaps as the result of a document preprocessing step.

Step D2: Segment Documents. Each document type category consists of one or more pages. The AD #1 algorithm requires single-page documents as input. Therefore, each incoming document type is separated into individual pages with all accompanying teeth view extracts stored as individual pages. The extracted views will optionally be normalized, e.g. by deskewing.

Step D3: Compute Hash Codes. A hash code is computed for each page generated by the document segmentation process and the Hamming Scorecard computed for the entire set of (new, incoming) pages. These codes and the resulting Hamming distances are the basis of the Reference Database search for matching or similar documents previously submitted as part of a claim.

Step D4: Evaluate Match Degree. The degree to which each incoming page matches a previously submitted and stored page can be read from the Scorecard. Exact matches correspond to a Hamming distance of zero while visually similar pages correspond to small, positive Hamming distances. A threshold value for what will be regarded as a significant image match is determined by the algorithm's user, with regard to the business logic being implemented.

Steps D5|D6: End Detection or Initiate Manual Review. When the Hamming distance of a page (available from the Scorecard) is less than or equal to the threshold, then the incoming claim is prioritized for human review since an indication of a match suggests that one or more pages may have been submitted previously in conjunction with another claim.

When the Hamming distance of a page is greater than the threshold value, the anomaly detection process concludes.

Alternative Embodiments. Several different forms of algorithm enhancement may be employed. Performance improvements can be achieved through additional experimentation with alternative hash code algorithms and their parameterizations. The existing implementation of AD #1 has been tested on a large set of dental insurance claims and found to perform well over the entire set of documents in that the hash code representation of each document type is efficient in terms of (1) storage space, (2) search time, and (3) robustness of the matching processes. The use of additional alternative hash code algorithms will likely improve real-world performance.

Specific forms of insurance fraud review can be enhanced by the use of tailored business logic. Two types of fraud inquiry of particular interest are inter-organizational (e.g., small dental practices) and cross-organizational (e.g., cooperating community organizations).

The significance/meaning of any given document match is established by the business logic employed by a particular claim assessment process. Various assessment process alternatives can be specified by using different or more elaborated business logics. Future work will examine the algorithmic tradeoffs involved in employing different business logics in conjunction with AD #1 as well as establish best practices for employing AD #1 in specific types of fraud inquiry. Currently AD #1 is restricted to searching for duplicate documents that are radiographs or photographs using both exact and inexact/similarity matching, or dental forms and probe charts using exact matching. Future work will focus on extending the inexact methods to ADA-approved claim forms and probe charts.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method performed on a processor for detecting duplication anomalies in a set of patient dental insurance records submitted as part of a dental insurance claim, said method comprising:
providing at least some of the patient dental insurance records as digital document(s);
generating at least one hash code representing each digital document from the provided patient dental insurance records;
calculating a Hamming distance between the generated document hash code and each of a plurality of hash codes in a database which includes hash codes representing documents from previous dental insurance claims; and
flagging the dental insurance documents for further review if the calculated Hamming distance between a document hash code and the database hash code differ by less than a threshold amount.

2. A method as in claim 1, wherein the patient dental insurance records include radiographic images.

3. A method as in claim 2, wherein the radiographic images contain multiple teeth view images, further comprising extracting one or more individual tooth images and generating the hash code for each individual tooth image.

4. A method as in claim 3, wherein the radiographic images are selected from a group consisting of a bitewing image, a periapical image, or a panoramic image.

5. A method as in claim 1, wherein providing the dental insurance records comprises providing additional patient documents including at least some of a patient probe depth-chart, patient correspondence, and one or more patient photographs as digitized documents.

6. A method as in claim 1, further comprising classifying the hash code for each digitized document according to a type of dental insurance record so that the hash code for any patient document can be compared only against hash codes for similar document types in the database.

7. A method as in claim 1, wherein providing at least some of the patient dental insurance records comprises digitizing at least some of dental insurance forms and images.

8. A method as in claim 1, wherein at least some of the patient dental insurance records are in a digitized format when provided.

9. A method for establishing and maintaining a reference database including hash codes representing sets of patient dental insurance records submitted as part of a dental insurance claim, said method comprising:
providing at least some of the patient dental insurance records as digital document(s);
generating at least one hash code for representing each digital document from the provided patient dental insurance records; and
saving the generated hash codes in the reference database.

10. A method as in claim 9, wherein the patient dental insurance records include radiographic images.

11. A method as in claim 10, wherein the radiographic images contain multiple teeth view images, further comprising extracting one or more individual tooth images and generating the hash code for each individual tooth image.

12. A method as in claim 11, wherein the radiographic images are selected from a group consisting of a bitewing image, a periapical image, or a panoramic image.

13. A method as in claim 9, wherein providing the dental insurance records comprises providing additional patient documents including at least some of a patient probe depth-chart, patient correspondence, and one or more patient photographs as digitized documents.

14. A method as in claim 9, further comprising classifying digitized documents according to a type of dental insurance record so that the hash code for any patient document can be stored with similar document types in the reference database.

15. A method as in claim 9, wherein providing at least some of the patient dental insurance records comprises digitizing at least some of dental insurance forms and images.

16. A method as in claim 9, wherein at least some of the patient dental insurance records are in a digitized format when provided.

17. A method performed on a processor for detecting duplication anomalies in a set of patient dental insurance records submitted as part of a dental insurance claim, said method comprising:
calculating Hamming distances between hash codes representing individual documents from the set of patient dental insurance records and hash codes from a reference database established and maintained as set forth in any claim 9; and
flagging the dental insurance forms for further review if the calculated Hamming Distance between the hash code of the submitted patient insurance records and the hash codes stored within the reference database differ by less than a threshold amount.

18. A reference database comprising a plurality of hash codes representing a plurality of sets of patient dental insurance records submitted as part of a plurality of dental insurance claims.

19. The reference database of claim 18, wherein said reference database is established and maintained by:
providing at least some of the patient dental insurance records in a form including at least one digitized document;
generating at least one hash code for each digitized document;
saving the generated hash code(s) in the reference database.
